(19) United States
(12) Patent Application Publication
(10) Pub. No.: US 2003/0115085 A1
(43) Pub. Date: Jun. 19, 2003

(54) Management system and method

(75) Inventor: Kazuhiko Satoh, Tokyo (JP)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Allied Telesis Kabushiki Kaisha, Tokyo (JP)

(21) Appl. No.: 10/190,896

(22) Filed: Jul. 8, 2002

(30) Foreign Application Priority Data
Dec. 13, 2001 (JP) 2001-380372

(51) Int. Cl. G06F 17/60

(52) U.S. Cl. 705/5; 705/1

(57) ABSTRACT

A management system for managing a facility that is available to users includes a network provided in the facility and connectible to a terminal, a communication part for communicating with the terminal through the network, a first memory for storing first information on an availability and status of use of the facility and service associated with the facility, the first information being able to be updated, and a control part for allowing the user of the terminal to access first information.

### BACKGROUND OF THE INVENTION

The present invention relates generally to systems for providing information through a network and an interconnecting device connected to the network. The present invention is suitable for an information providing system for use with a facility, such as a hospital, a beauty salon, and a health club, a restaurant, etc. The facility lays out a computer network, such as a LAN (Local Area Network), and the information providing system provides information including a waiting time for use of the facility and which enable booking of the use of the facility.

Along with recently spread LANs and WANs (Wide Area Networks), a large number of network devices, such as personal computers (“PCs” hereinafter), hubs, switches, and routers (hubs etc. are often called “agents”) have been connected to a network and its subnet(s) for frequent information sharing and communications. For example, a facility such as a hospital has laid out a network, e.g., a LAN, and used a management server to manage security in the hospital and the network. The network promotes information sharing, and manages information on medical examinations and treatments (which are simply referred to as “treatments” hereinafter unless otherwise specified in this application) in each department (e.g., the department of surgery and the internal department), prescription, inpatients and hospitalization, and hospital facilities including medical offices, operation room(s), and X-ray room(s).

Such a conventional hospital assigns an in-hospital administrator to handle managements of the LAN, but this system does not expect patients' uses. For example, a patient cannot usually obtain information on the waiting time for his medical treatment and his chart. A certain hospital may provide a counter at the reception of each department so as to indicate the number of waiting persons, but once the patient goes away from the department, he cannot obtain the waiting information. As a result, a patient often feels that he has waited for a longer time than expected in order to take a medical treatment, to finish paying the bill at the cashier, and to receiving a prescription medicine from a pharmacy or dispensary. Indeed, a patient often spends about half a day or a whole day in a hospital. One reason of this delay results from a manual handling of a paper, patient's chart. For example, the patient's turn for a medical treatment is improperly changed as a result of erroneous handling of his chart. When a medical treatment finishes, the chart is not forwarded to the cashier immediately but is often stacked in a rack. Then, multiple charts are delivered at one time to reduce the number of reciprocations between the medical department and the cashier in the hospital. An operator in the cashier spends much time to manually prepare a bill by checking a chart. After all, a patient cannot expect how much time he should spend in the hospital. Moreover, it is not until a patient goes there that he notices that the doctor who is in charge of him is absent from the hospital.

### BRIEF SUMMARY OF THE INVENTION

Accordingly, it is an exemplified object of the present invention to provide a management system and method for providing users with efficient use of a facility and service associated with the facility.

In order to achieve the above objects, a management system as one aspect of the present invention for managing a facility that is available to users includes a network provided in the facility and connectible to a terminal, a communication part for communicating with the terminal through the network, a first memory for storing first information on an availability and status of use of the facility and service associated with the facility, the first information being able to be updated momentarily, and a control part for allowing the user of the terminal to access first information: According to this management system, each user (e.g., a patient) may previously grasp the availability and the crowdedness of the facility (e.g., a certain department in the hospital) and service associated with the facility (e.g., a medical treatment by a certain doctor). For example, the first information includes the number of waiting persons and/or estimated waiting time for each department in a hospital in the specific date and time, an availability of each doctor in a specific date and time, an availability of each facility in the hospital in the specific date and time, a description of symptoms, a description of a prescription medicine, or frequent asked questions. The terminal may be provided in the facility, or each user may possess the terminal connectible to the network. As a result, a patient may check if a target doctor is available on a specific date and time beforehand, and thus he can avoid a case where it is not until he goes there in that time that he notices that the doctor who is in charge of him is absent from the hospital.

The management system may further include a second memory for storing second information on identification of the users, and an authentication part for authenticating a person who uses the terminal, based on the second information in the second memory and third information entered by the person through the terminal, wherein the control part allows the person to access the first information when the authentication part authenticates the person. Thereby, an unauthorized use of the network and facility may be prevented. The control part may serve as the authentication part.

Each user may have an information record carrier (e.g., an IC card) for storing the third information, and the terminal includes a drive (e.g., an IC card drive) for reading the third information from the information record carrier. Therefore, an authorized person who does not have the information record carrier cannot use the network and facility. The information record carrier may store fourth information on a result of use of the facility and service associated with the facility (e.g., a result of a medical treatment or a chart). Thereby, the user may confirm the use of the facility and service. In some case, the information record carrier may store billing information including a fee of using the facility and/or the service associated with the facility.

The third information may include a condition of the user (e.g., a height, weight, sex, and age). For example, the users are patients, and the condition of the user includes symptoms of the user (e.g., a headache, fever, and allergy to a medicine). The control part may accept a reservation, a cancellation of the reservation and a change of the reservation by the user of the facility and service associated with the facility, and update the first information to reflect the reservation, the cancellation of the reservation, and the change of the reservation. Thereby, the users may make a reservation, cancel or change the reservation by themselves at their desired time and date. This system is laborsaving for the facility since the users do not use a worker of the facility. In addition, the users always see the updated first information. The term “reservation” in accompanying claims covers reservations of all future use.

Each user may have an information record carrier for storing fourth information on a result of use of the facility and service associated with the facility by the user (e.g., a result of a medical treatment or a chart), and wherein the management system may further include a drive for writing the fourth information in the information record carrier after use of the facility and service associated with the facility by the user, a cashier part for creating billing information based on the fourth information obtained through the network. The cashier part may receive the fourth information from the user's information record carrier or through the network. In either case, when or before the user reaches the cashier part, the cashier part may receive the fourth information and prepare the billing information. The management system may further include a third memory for storing a table that correlates expenses and use of facility and service associated with the facility, wherein the cashier part automatically calculates the billing information referring to the third memory. Since the billing information is created automatically and promptly, the user does not have to spend much time. The information record carrier may further store fifth information on an account of the user (e.g., bank or credit card information or electronic money account information), and the cashier part bills the account based on the fifth information. Since the billing information is automatically charged to the user's account, the user does not have to spend much time at the cashier.

Each user may be a patient and has an information record carrier, the information record carrier storing fourth information on a result of a medical treatment and a prescription medicine, the facility including a pharmacy, wherein the management system may further include a drive, provided in the pharmacy, for reading the fourth information in the information record carrier to provide the prescription medicine. Since the prescription information has been already stored in the information record carrier, the cashier, for example, does not have to print out the prescription and provides the user with it. As a result, the user may quickly go to the pharmacy and receive the prescription medicine.

The control part may control connection status and traffic of the network. Thereby, the control part provides a centralized administration of the network. The terminal logs in the network in a wireless manner. For instance, the terminal may use a cellular phone, infrared communication, or a bluetooth. Thereby, the user does not have to go to the facility to access the first information, and may log in the network from the outside.

A management method of another aspect of the present invention for managing a facility that is available to users includes the steps of storing first information on an availability and status of use of the facility and service associated with the facility by the users, the first information being able to be updated momentarily and accessible to each of the users through a terminal and an information record carrier that stores the third information, storing second information on identification of the users, authenticating a person who uses the terminal, based on the second information and third information entered by the person through the terminal and the information record carrier, allowing the person to access the first information when the authentication step authenticates the person. This method also exhibits similar operations to those of the management system. A program that enables a computer to execute the above management method is also constitutes another aspect of the present invention.

The management method may further include the steps of accepting a reservation, a cancellation of the reservation and a change of the reservation by the person to reserve the facility and service associated with the facility on a specific date and time when the authentication step authenticates the person, and updating the first information to reflect the reservation, the cancellation of the reservation, and the change of the reservation. Thereby, the users always see the updated first information.

A management device of another aspect of the present invention connected to a network provided in a facility that is available to users, and a terminal through the network, the network being connected to a first memory for storing first information on an availability and status of use of the facility and service associated with the facility, the first information being able to be updated momentarily includes a second memory for storing second information on identification of the users, an authentication part for authenticating a person who uses the terminal, based on the second information in the second memory and third information entered by the person through the terminal, and a control part allows the person to access the first information in the first memory when the authentication part authenticates the person. The management device maintains security of the network, and enables each user (e.g., a patient) to grasp the availability and the crowdedness of the facility (e.g., a hospital and a certain department in the hospital) and service associated with the facility (e.g., a medical treatment by a certain doctor).

Other objects and further features of the present invention will become readily apparent from the following description of preferred embodiments with reference to accompanying drawings.

### BRIEF DESCRIPTION OF THE DRAWINGS

FIG. 1 is a structural view of a management system of the present invention.

FIG. 2 is a schematic block diagram of a management device in the management system shown in FIG. 1.

FIG. 3 is a schematic block diagram of a structural example of a memory shown in FIG.2.

FIG. 4 is an exemplary table stored in a personal information database shown in FIG.3.

FIG. 5 exemplarily shows information stored in an IC card shown in FIG. 2.

FIG. 6 is a schematic block diagram of an interconnecting device in the management system shown in FIG. 1.

FIG. 7 is a schematic view of the interconnecting device shown in FIG. 6.

FIG. 8 is an exemplary timetable.

FIG. 9 is a schematic block diagram of a network device in the management system shown in FIG. 1.

FIG. 10 is a flowchart for explaining an operation of the management system shown in FIG. 1.

### DETAILED DESCRIPTION OF THE PREFERRED EMBODIMENTS

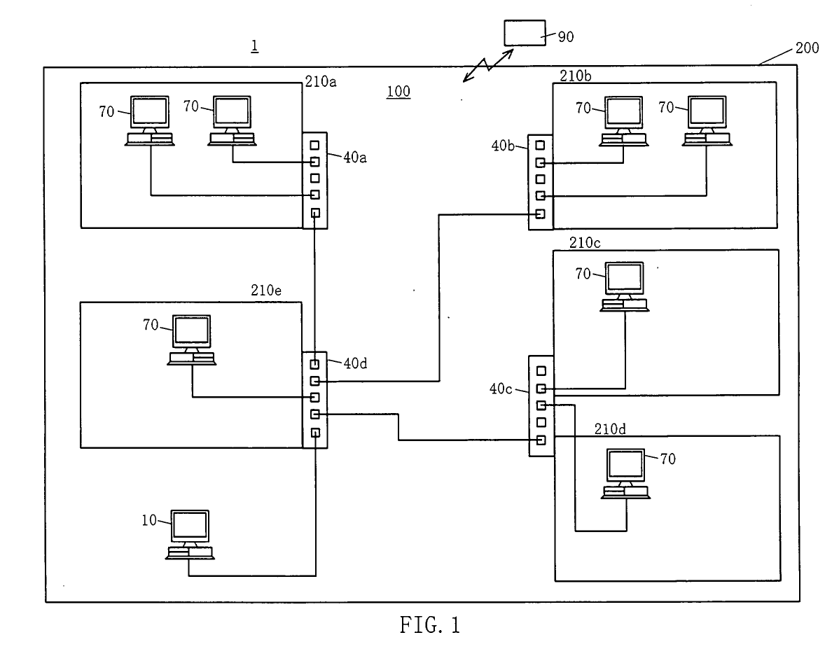

A description will now be given of a management system 1 of the present invention, with reference to the accompanied drawings. Here, FIG. 1 is a structural view of the management system 1. The inventive management system 1 includes a network 100 including a management device 10, interconnecting devices 40 (i.e., 40a-40d), and a plurality of network devices 70, and is applied to a hospital 200. Optionally, the network 100 may communicate with a terminal 90.

Specifically, one or more network devices 70 are provided each department 210 (e.g., a department of surgery 210a, an internal department 210b, a department of inspection 210c, a pharmacy or dispensary 210d, a reception 210e) in the hospital 200, and concentrated to the interconnecting devices 40a-40d in the department 210. The interconnecting device 40 does not have to be provided for each department 210, and multiple departments (e.g., the department of inspection 210c and the dispensary 210d) may share one interconnecting device 40 like the interconnecting device 40c, as shown in FIG. 1. FIG. 1 shows seven network devices 70 for exemplary purposes only, and the present invention does not limit the number of network devices 70.

The management device 10 controls connection statuses and traffic in the network devices 70 and other devices, and thereby manages the configuration, performance, security, and billing of the network 100.

In the configuration management, the status conditions of devices in the network 100 are monitored and its operation is controlled. The configuration management also includes an acquisition of a firmware version for each device. This allows a network administrator to grasp the current network configuration of network devices connected to the network 100 and its subnet(s). An administrator evaluates the performance of each device in the network 100 in the performance management. The performance management allows the network administrator to monitor conditions of traffic of each device to see the network activities as to whether an abnormal packet is being transmitted, whether the server acts properly, or the like. In the security management, the use of resources (information and devices) in the network is restricted and controlled to encourage authorized use of the resources. The network administrator periodically investigates users' access logs to find out an unauthorized use, or to prevent an unauthorized user from invading the network. In the billing management, the use logs of the resources in the network are recorded for each user. The network administrator grasps the duty cycle of the network resources for each user to warn a user who abuses the resources or to use as materials for better services.

Although not described in detail, the management device 10 manages the network 100 using a Dynamic Host Configuration Protocol (“DCHP”) for providing the interconnecting devices 40 and network devices 70 with communication parameters for identifying them in the network 100. The communication parameter includes an IP address, a subnet mask, a default gateway. This network management may use any technique known in the art, and thus a detailed description will be omitted. A method for providing the communication parameter may use any technique known in the art including the management device 10 assigning the communication parameter to the network device 70 when recognizing power on of the network device 70.

The management device 10 in the present embodiment is exemplarily a desktop PC, to which an IC card drive 20 is attached externally or internally. A contact-type IC card 30 is used for the IC card drive 20, but the noncontact-type IC card is not excluded from application to the present invention. Further, the present invention is also applicable to information record carrier other than the IC card, such as a PC card, and a memory card.

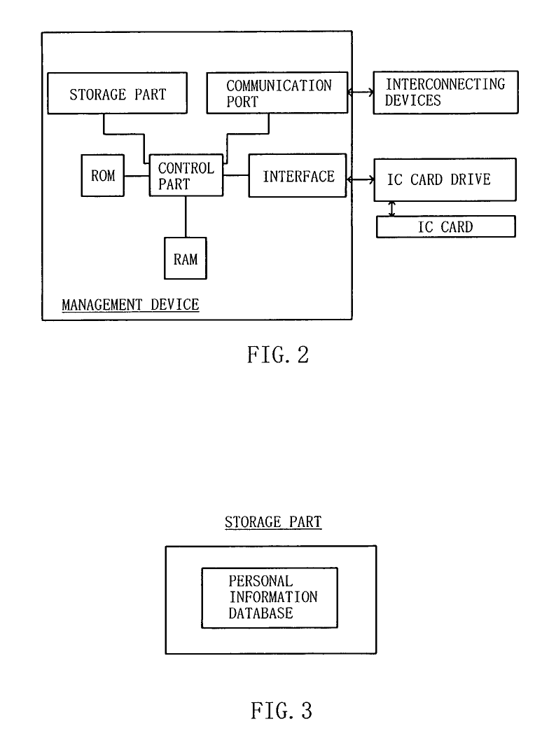

The management device 10 includes, as shown in FIG. 2, a control part 11, a communication port 12, a RAM 13, a ROM 14, a storage part 15, an interface 16, and the IC card drive 20. Here, FIG. 2 is a schematic block diagram of the management device 10. In FIG. 2, input/output devices (e.g., a keyboard, a mouse or other pointing devices, and a display) attached to the management device 10 are omitted. Through the input/output device, an operator of the management device 10 may control the IC card drive 20, input data of various kinds in the storage part 15, and download necessary software into the RAM 13, and ROM 14 or storage part 15.

The control part 11 covers a broad range of processors such as a CPU and an MPU regardless of its name, and controls each section in the management device 10. If necessary, the management device 10 may be connected to a host (not shown), and the control part 11 may communicate with the host. The control part 11 stores in the IC card 30 information in the storage part 15 through the IC card driver 20. Moreover, the control part 11 authenticates information sent from the interconnecting device 40 with reference to a personal information database 15a in the storage part 15.

The communication port 12 may be an LAN adapter connected to the interconnecting devices 40, and a USB port or IEEE 1394 port for providing connections to the Internet (if necessary, via an Internet Service Provider (ISP)) via a modem, or a terminal adapter (TA) through the public telephone network, ISDN, or various types of dedicated lines. In some embodiment, the communication port 12 may be connected to the terminal 90 through the Internet.

The RAM 13 temporarily stores data to be read from the ROM 14 and storage part 15, data to be written in the storage part 15, and the like. The ROM 14 stores various kinds of software and firmware required for operations of the control part 11, and other types of software.

The storage part 15 stores, as shown in FIG. 3, the personal information database 15a. Here, FIG. 3 is an exemplary block diagram of the contents of the storage part 15 shown in FIG. 2.

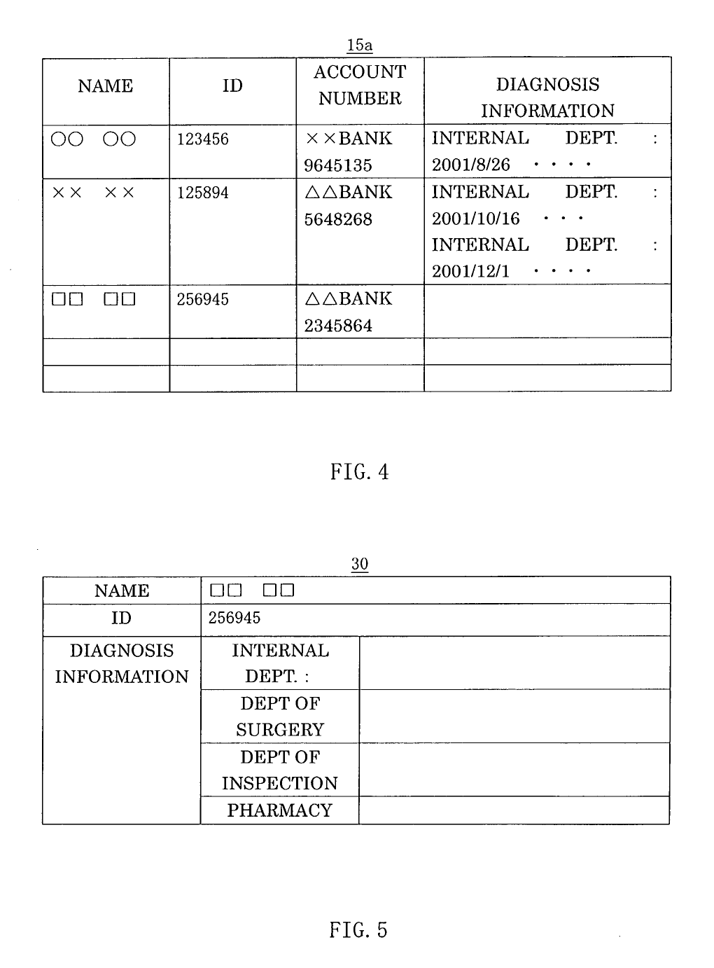

The personal information database 15a stores information including identifications of patients of the hospital 200, and is used to authenticate a person who attempts to use the hospital 200 through communications with the interconnecting devices 40. As apparent from the following operation, an authentication of a patient uses reading of his IC card 30. Instead of distributively providing the database 15a in the network 100, the management device 10 holds the database 15a and advantageously provides a centralized management for security purpose. The personal information database 15a stores a table including a name, ID, an account number, diagnosis information, etc., and thus the table may further include a social security number, a date of birth, an address, a phone number, a mother's maiden name, a pet's name, and a password, any other identifying information. Here, FIG. 4 is an exemplary table stored in the personal information database 15a shown in FIG. 3.

The “name” is a field to store a patient's name. The “ID” is a field to store a patient's identifier. The ID is preferably unique in the hospital 200, and sequentially assigned by an administrator at the time of initial medical treatment or hospitalization in the ascending or descending order (for example, it is a numerical combination of six digits, such as 0123456). The “account number” is account information of patient's bank account, credit number, electronic money account, and the like from which a hospital bill is automatically deducted. This field, not necessarily provided, is used to pay the bill in the hospital 200 and assists in smooth settlement in the hospital 200. In addition, such a system is timesaving for the patient and conveniently eliminates the necessity of the patient to bring lots of money to the hospital 200. The “diagnosis information” is a field to store a patient's diagnosis result to be filled in a chart.

As discussed above, the present invention does not restrict the personal information database 15a from including additional fields. Therefore, an administrator may add or delete arbitrary fields or partially change the fields in FIG. 4 according to environments to which the inventive management system is applied.

The storage part 15 may store a timetable 45 stored in the storage part 45 in the interconnecting device 40, if necessary, through a communication between the management device 10 and each interconnecting device 40. When the timetable 45a is stored in the storage part 15, the management device 10 may provide information, which would be otherwise provided by interconnecting devices 40.

The interface 16 is, for example, a USB or a parallel port, and connects the management device 10 to an external device as the IC card drive 20 in this embodiment. The interface 16 includes any interface irrespective of a type of data transmission method, such as parallel and serial systems, and a type a connection medium, such as a radio and wire transmissions.

The IC card drive 20 reads information from and writes information on the IC card 30. In this embodiment, the control part 11 records part or all of the personal information database 15a output through the interface 16 down onto the IC card 30. The present invention does not limit the information record carrier to the IC card 30, but may apply any other information record carrier and drive for driving the information record carrier. The IC card drive 20 may use any technique known in the art, and thus a detailed description thereof will be omitted.

The IC card 30 is issued to a patient and inpatient, and serves as a consultation card or ticket to take a diagnosis in the hospital 200 and a chart. As described later, the patient makes the IC card drive 20 in the interconnecting device 40 read the IC card 30, so as to apply for today's medical treatment, make a reservation, cancel and change the reservation of a medical treatment, and obtain an estimated waiting time and/or the number of waiting persons. In this embodiment, the terms “registration” and “application” are sometimes used to reserve a today's medical treatment, and discriminated from the term “reservation” that is used to reserve a non-today's medical treatment. In this embodiment, the “reservation” of a medical treatment is available when the target medical treatment/examination time is scheduled at least 24 hours after the current time. The “registration” and “application” usually are based on a principle “first come, first served”, while the “reservation” designates a specific date and time for a medical treatment. The registration may be made by a patient or a reception in the hospital 200. A doctor reads the IC card 30 through an IC card drive 70 in the network device 80, so as to read patient's chart information stored in the IC card 30, and to write new diagnosis information in the IC card 30.

The IC card 30 stores various fields that exist in the personal information table 15a for the patient. As shown in FIG. 5, the IC card 30 exemplarily stores a name, an ID, diagnosis information, etc. Here, FIG. 5 is an example of the personal information table 15a.

The IC card 30 may use unique external appearance to differentiate stored information in this embodiment. For example, the IC card 30 may indicate a letter, design, and a color or a combination of them, depending upon an outpatient and an inpatient, directly (for example, by providing a direct indication on a case of the IC card 30) or indirectly (for example, by labeling the case of the IC card 30).

The IC card 30 is a general term that covers a smart card, an intelligent card, a chip-in card, a microcircuit (microcomputer) card, a memory card, a super card, a multi-function card, a combination card, and the like. In addition, the IC card of the present invention is not limited to a card-shaped medium, but includes any medium which is, for example, of the size of a postage stamp or smaller, i.e., very small-size one, or shaped like a coin, etc.

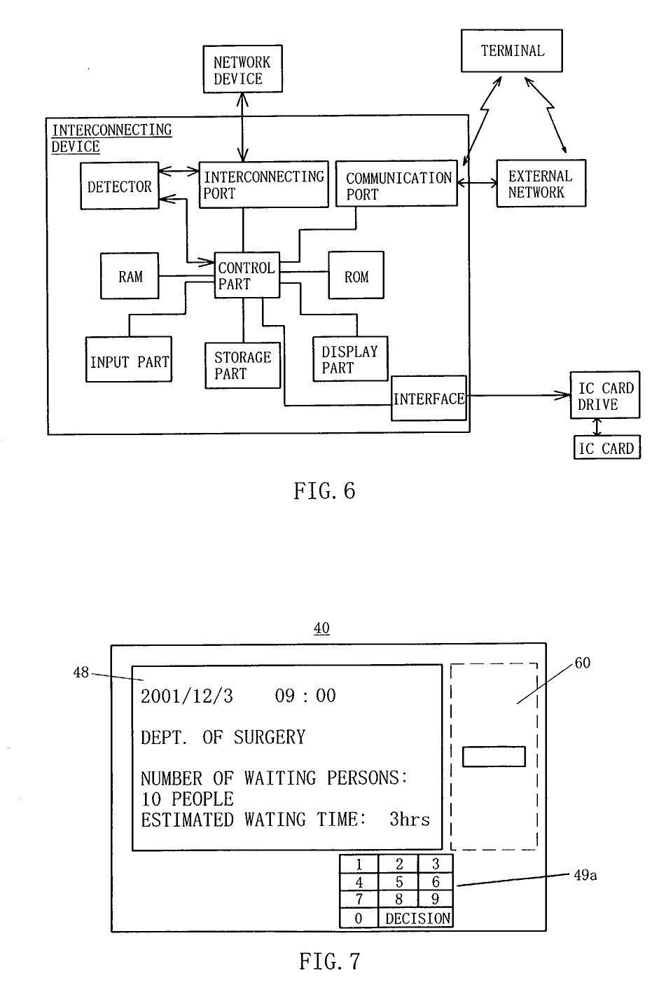

The interconnecting device 40 in this embodiment covers an interconnecting network device for connecting the interconnecting device 40 and the network device 70 to the Ethernet, and an information providing device used for a patient. The interconnecting device 40 includes, for example, a hub, a switch, a router, any other concentrator, a repeater, a bridge, a gateway device, a PC, and a wireless interconnecting device (e.g., an access point as a interconnecting device for wireless LAN). For example, the interconnecting device 40 is a combination of the PC and the hub, although FIG. 1 does not show an information-provider aspect of the interconnecting device 40. Instead, FIG. 6 shows the information-provider aspect of the interconnecting device 40. Of course, the aspect of the information provider may be detached from the interconnecting device 40. In that case, PCs, etc. would be connected to the interconnecting device 40 or part of the network device 70 would be open to patients.

The present embodiment uses the Ethernet as a typical LAN for the network 100. The Ethernet is a LAN in a bus topology, and includes 10Base-T, 100Base-TX, Gigabit Ethernet, and the like. However, the present invention is applicable to other types of LAN (e.g., Token Ring), and networks other than LAN such as WAN, MAN (Metropolitan Area Network), private network, the Internet, commercial dedicated lines network (e.g., America Online), and other networks.

In this embodiment, the interconnecting device 40 provides a patient with a guidance relating to use of the hospital 200, such as information on waiting time, and accepts a registration, a reservation, a change or cancel of the reservation of a medical treatment. In making a reservation, the interconnecting device 40 enables the user to enter additional information for doctor's review. Such information may be patient's condition, such as a height, weight, sex, age, and symptoms including a headache, fever, allergy to a medicine, etc. The information provided by the interconnecting device 40 relates to an availability and status of use of facilities in the hospital 200 and service associated with the facility, and may cover the number of waiting persons and/or estimated waiting time for each department in a hospital in the specific date and time, an availability of each doctor in a specific date and time, an availability of each facility in the hospital in the specific date and time, a relationship between symptoms and proper departments a description of a prescription medicine, and other frequent asked questions.

The interconnecting device 40 includes, as shown in FIGS. 6 and 7, a control part 41, an interconnecting port 42, a RAM 43, a ROM 44, a storage part 45, a detector 46, a communication port 47, a display part 48, an input part 49, an interface 50, and an IC card drive 60. Here, FIG. 6 is a schematic block diagram of the interconnecting device 40. FIG. 7 is a schematic view of the display part 48. Through the input part 49, an operator of the interconnecting device 40 may input various kinds of data in the storage 45, and download necessary software into the RAM 43, and ROM 44 and storage part 45. While the interconnecting device 40 has a built-in IC card drive 60 in this embodiment, the IC card drive 60 may be provided external to the interconnecting device 40.

The control part 41 broadly covers processors including a CPU and a MPU, regardless of its name, and controls each section in the interconnecting device 40. The control part 41 in this embodiment reads information from the IC card 60, calculates a waiting time for a medical treatment based on a control method stored in the storage part 45, and communicates with other interconnecting device 40 to obtain waiting information. Although not described in detail, the control part 41 communicates with the detector 46, and provides the management device 10 with information to identify the network devices 70, and relays the management by the management device 10 to the network devices 70.

The interconnecting port 42 is a communication port connected to each network device 70 and the other interconnecting device 40 by a cable or the like. The RAM 43 temporarily stores data to be read from the ROM 44 and storage part 45, data to be written in the storage part 45, and the like. The ROM 44 serves to store various kinds of software and firmware necessary for operations of the control part 41, and other types of software.

The storage part 45 stores a control method to be executed by the interconnecting device 40. The management method as a program may be distributed as an independent commodity. Therefore, the program may be sold in a CD-ROM or other record carrier, or distributed and updated online via a network such as the Internet.

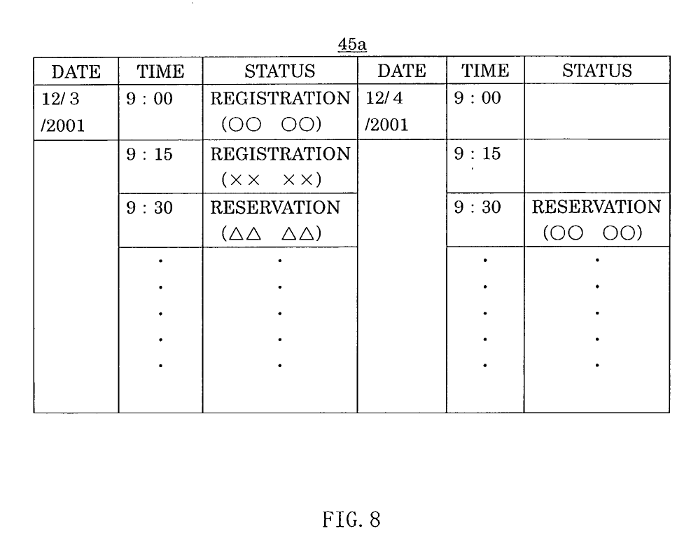

The storage part 45 further stores a timetable 45a as shown in FIG. 8. Here, FIG. 8 is an example of timetable 45a. The timetable 45a stores information on a date and time for the department(s) or area(s) 210 for which the interconnecting device 40 is provided. In this embodiment, the timetable 45a may store a relationship between time and reserved, registered and available medical treatments. The instant embodiment exemplarily forms the timetable 45a every quarter. When a plurality of areas 210 (e.g., areas 210c and 210d) share one interconnection device 40, the storage part 45 stores a plurality of timetables 45a corresponding to these areas 210.

The detector 46 detects power-on of the network device 70 by communicating with the interconnecting port 42, and notifies the control part 41 of the detection result. Since the detector 46 uses any structure known in the art, for example, a structure that compares the voltage of the interconnecting port 42 with a specific slice level for detection purposes, a detailed description of the detector 46 will be omitted. The detector 46 is used for the management of the network 100 by the management device 10.

The communication port 47 may be an LAN adapter for providing the interconnecting device 40 with a connection to the other interconnecting device 40 or the Ethernet, and a USB port or IEEE 1394 port for providing connections to the Internet (if necessary, via an Internet Service Provider (ISP)) via a modem, or a terminal adapter (TA) through the public telephone network, ISDN, or various types of dedicated lines. The communication port 47 is the same as the interconnecting port 42 in some embodiment, but connects the interconnecting device 40 to an external network, such as a telephone network and the ISDN, which is connectible to the Internet in the instant embodiment.

The display part 48 may be a known display, such as a CRT and a TFT, and displays information on the number of waiting persons and reserved and registered medical treatments for patients. Referring to the display part 48, a patient may decide to change, register or reserve his turn, and grasp the waiting time for a medical treatment at another department. As stated above, the display part 48 may indicate additional information that has nothing to do with the waiting information, such as a description of a prescription medicine, and thus a patient may confirm an ingredient of his prescription medicine.

The input part 49 includes a known input means, such as a keyboard, pointing device, switch and button. A user (who is a patient or administrator) may manipulate the input part 49 to switch an item (or mode) of a menu, register or reserve a medical treatment. As shown in FIG. 8, the input part 49 in this embodiment includes ten key 49a. The input part 49 may be combined with the display part 48 into a touch panel.

The interface 50 is, for example, a USB or a parallel port, and connects the interconnecting device 40 to an external device as the IC card drive 60 in this embodiment. The interface 50 includes any interface irrespective of a type of data transmission method, such as parallel and serial systems, and a type a connection medium, such as a radio and wire transmissions.

The IC card drive 60 reads information from and writes information into the IC card 30. As shown in FIG. 8, the IC card drive 60 has an IC card insertion opening 61, through which the IC card 30 is inserted into and ejected from the IC card drive 60. Like the IC card drive 20, the IC card drive 60 may use any technique known in the art, and thus a detailed description thereof will be omitted.

The network device 70 is a device connected to the network 100 and managed by the management device 10. In this embodiment, only doctors, nurses and other workers in the hospital 200 use the network devices 70. In an alternate embodiment, however, the network device 70 may also serve as an information providing device open to patients and/or inpatients. The network device 70 includes a network device, such as a hub, a switch, a router, any other concentrator, a repeater, a bridge, a gateway device, a PC, a server, a wireless interconnecting device (e.g., an access point as a interconnecting device for wireless LAN), and a game machine having a communication function. The instant embodiment connects seven network devices 70 to the network 100 and its subnet(s). The network device 70 is implemented as a PC in this embodiment, and used by doctors and other workers in the hospital 200. The network device 70 may record patient's chart information in the IC card 30, access the personal information database 15a in the management device 10, communicate with other network device 70, and perform various work using application software in the PC.

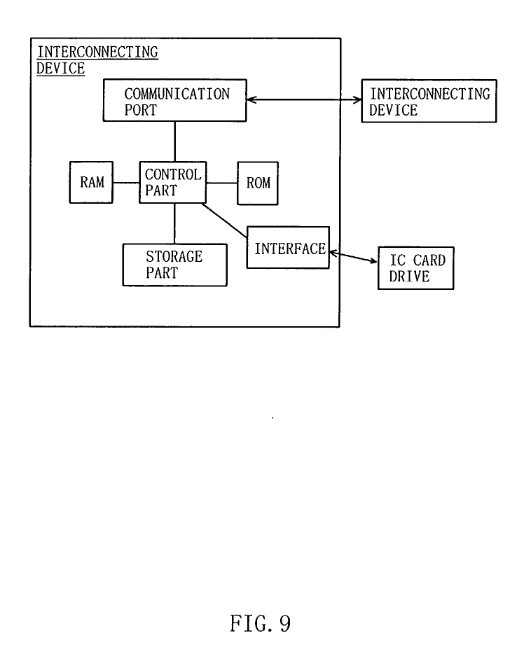

The network device 70 includes, as shown in FIG. 9, a control part 71, a communication port 72, a RAM 73, a ROM 74, a storage part 75, an interface 76, and an IC card drive 80. Here, FIG. 9 is a schematic block diagram of the network device 70. FIG. 9 also omits the input/output devices provided with the network device 70 for simplicity purposes. Through the input device, an operator of the network device 70 may input various kinds of data in the storage part 75, and download necessary software into the RAM 73, and ROM 74 and storage part 75. The IC card drive 80 may be provided inside or outside the network device 70 in FIG. 9.

The control part 71 covers a broad range of processors such as a CPU or an MPU regardless of its name, and controls each section in the network device 70. The control part 71 may read information from and write information onto the IC card 30 through the IC card drive 80. The control part 71 may send information read from by the IC card drive 80 to the management device 10 and/or the interconnecting device 40 through the communication port 72.

The communication port 72 may be an LAN adapter for establishing a connection to the network 100, and a USB port or IEEE 1394 port for providing connection to the Internet (if necessary, via an Internet Service Provider (ISP)) via a modem, or a terminal adapter (TA) through the public telephone network, ISDN, or various types of dedicated lines. The RAM 73 temporarily stores data to be read from the ROM 74 and storage part 75, data to be written in the storage part 75, and the like. The ROM 74 stores various kinds of software and firmware necessary for operations of the control part 71, and other types of software.

The storage part 75 stores a communication parameter and a configuration program. The configuration program is a program to receive communication parameters corresponding the DHCP and to set up them. Preferably, the storage part 75 of the network device 70 in the cashier 210e stores a table that correlates expenses and medical treatments. Since the IC card 30 stores chart information, the network device 70 at the cashier part may automatically calculate the billing information with reference to the storage part 75. Alternatively, the network device 70 in each department may automatically prepare the billing information when the doctor prepares the chart information. In this case, the IC card 30 also stores the billing information so that the patient may confirm both a relationship between the expense and medical treatment.

The interface 76 is, for example, a USB or a parallel port, and connects the network device 40 to an external device as the IC card drive 80 in this embodiment. The interface 76 includes any interface irrespective of a type of data transmission method, such as parallel and serial systems, and a type a connection medium, such as a radio and wire transmissions.

The IC card drive 80 reads information from and writes information on the IC card 30. Like the IC card drive 20, the IC card drive 80 may use any technique known in the art, and thus a detailed description thereof will be omitted.

The terminal 90 may be optionally provided as shown in FIGS. 1 and 6, and communicate with the network 100. A patient, a doctor and other workers may use the terminal 90 to access the network 100, e.g., the communication port 47 in the interconnecting device 40. The terminal 90 includes a portable or non-portable, wireless or radio communication device, for example, a cellular phone, a device having a bluetooth module, a device a Personal Digital Assistant (“PDA”), a game machine, and a device having an infrared function.

The bluetooth is a short-distant radio communication standard connecting PCs, peripherals, cellular phones, and information terminals to each other by a spread spectrum packet communication, and includes a synchronous transmission channel for audio transmission and an asynchronous transmission channel for data transmission. The connectable range for the bluetooth is about 10 m, but is extendable up to 100 m using an additional amplifier. The bluetooth module integrates a bluetooth, and includes a radio signal transmitter/receiver, a processor, a first converter, and a second converter. The radio signal transmitter/receiver transmits a radio signal that the bluetooth module has converted and receives the radio signal. The radio signal transmitter/receiver may apply any structure known in the art, for example, including a D/A converter, a low pass filter, a FM modulator, a burst modulator in the transmitter and an IF filter, an FM modulator, a threshold detector/clock recovery, a frequency hopping controller in the receiver. The processor controls each part in the bluetooth module, and made by a DSP (digital signal processor), for example, to control audio and image signals, links, packets, error corrections, securities, data randomization, etc.

The first converter converts a radio signal into audio information, and audio information into a radio signal. The second converter converts a radio signal into image information, and image information into a radio signal. The processor receives audio information to be supplied to the first converter, and image information to be supplied to the second converter. The processor transmits to the external device audio information converted by the first converter and image information converted by the second converter.

The terminal 90 does not have to use the wireless or radio communication. For example, when the terminal 90 is constituted as a PC connectible to the Internet, the terminal 90 may communicate with the communication port 47 in the interconnecting device 40 through the Internet. Alternatively, the terminal may communicate with the communication ports 12 and/or 72 through the Internet.

The terminal 90 enables the patient to check the timetable 45a without going to the hospital 200 or the target department in the hospital 200. In addition, the doctor may check his schedule anywhere in the hospital 200 by using the terminal 90 to access the timetable 45a. Preferably, the terminal 90 has a drive for reading an information record carrier. As stated above, the information record carrier is not limited to the IC card 30 and may use a PC card and other information record carriers.

A description will now be given of an operation of the inventive management system 1. First, an administrator creates the IC card 30 as a consultation ticket for a patient, and his personal information database 15a in the management device 10. The personal information database 15a is created, for example, at time of the first medical treatment. Information used to fill in the fields in the personal information database 15a may be collected from a patient electronically or non-electronically via the Internet, mail, facsimile, telephone, oral hearing when or before the patient visits the hospital 200. If necessary, the administrator may update or add the personal information database 15a.

The control part 11 prompts the administrator to enter a patient's name and store it in the “name” field in the personal information database 15a. The control part 11 stores an identification number after the “name” field is filled. The administrator may arbitrarily select a proper identifier and store it in the “ID” field, or the control part 11 may automatically assign a proper identifier, which is preferably unique in the hospital 200, according to a preset rule (e.g., in an ascending order of six digits).

As in the instant embodiment, the administrator may store, in the “account number” field, a bank account number, a credit card number, etc. for payment to a bill for a medical treatment and prescription medicine. Of course, there should be a predetermined agreement between the patient and a bank or credit card company for settlement. Thereby, the patient utilizes a bank's automatic deduction service for payment, and does not bring much money to the hospital 200. This system is also timesaving for the patient since the patient does not have to spend time to arduously wait at the cashier.

The management device 10 stores the personal information database 15a in the storage part 15, but the personal information database 15a does not have to be prepared by the management device 10 and another PC etc. may prepare the personal information database 15a.

The administrator obtains information belonging to the patient from the personal information database 15a stored in the storage part 15, and stores the information in the IC card 30 through the IC card drive 20 so that the patient may use the IC card 30. This initialization stores, in the IC card 30, minimum information for using the management system 1.

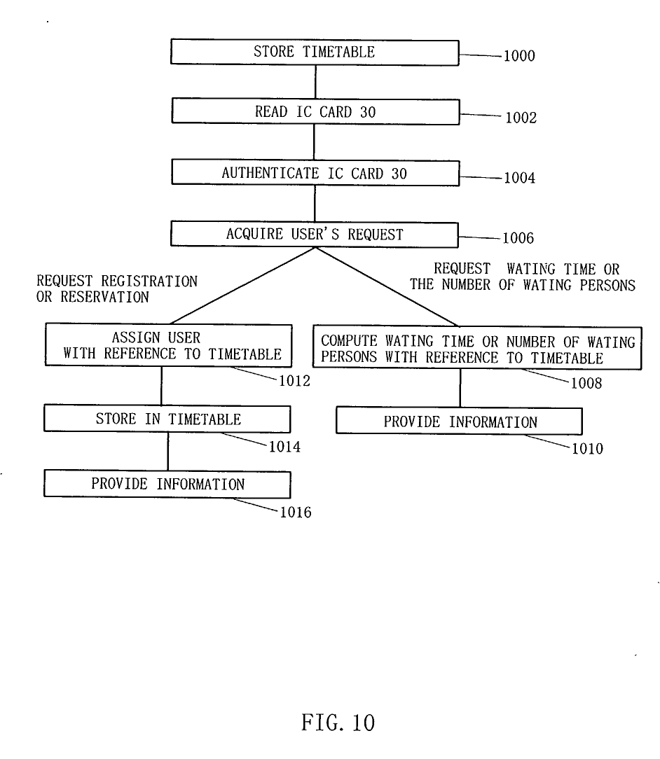

A description will now be given of a medical treatment using the inventive management system 1. Here, FIG. 10 is a flowchart for explaining an operation of the inventive management system 1. Now, a patient of the hospital 200 possesses the IC card 30 that stores predetermined information through the above operation. The storage part 45 in the interconnecting device 40 has stored the timetable 45a through the reservation and registration (step 1000).

The patient goes to the hospital 200 carrying the IC card 30, and uses the interconnecting device 40d at the reception 210e to apply for a today's medical treatment. Of course, the patient does not have to use the interconnecting device 40d, and he may use any one of the interconnecting device 40a-40d. For example, the patient may go to a target department and uses the interconnecting device 40 provided for the target department.

The control part 41 in the interconnecting device 40d (or one of the interconnecting devices 40a-40c) indicates a message “Please insert IC card.” for promote patient's utilization. This is an initial screen in the instant embodiment. The patient inserts his IC card 30 into the IC card insertion opening 61 in the IC card drive 60 in the interconnecting device 40. In response, the IC card drive 60 reads the inserted IC card 30 (step 1002). The information read by the IC card drive 60 is sent to the control part 41 in the interconnecting device 40d through the interface 50, whereby the control part 41 obtains the information stored in the IC card 30. The control part 41 transmits the read information to the management device 10 through the interconnecting port 42.

The management device 10 receives this information through the communication port 12, and the control part 11 obtains the information (and then stores it in the RAM 13 temporarily). When the control part 11 obtains the information from the interconnecting device 40d, it determines whether the information accords with information stored in the personal information database 15a in the storage part 15 so as to authenticate the IC card 30 and thus the patient (step 1004).

Specifically, the control part 11 refers to the personal information database 15a in the storage part 15, and retrieves the personal information database 15a using the received information, e.g., a name and/or ID. When finding both in the personal information database 15a, the control part 11 notifies, through the communication port 12, the interconnecting device 40d that the IC card 30 has been authenticated.

On the other hand, when not finding the information that accord with the information received from the interconnecting device 40d as a result of the search using the personal information database 15a, the control part 11 notifies the interconnecting device 40d that the IC card 30 has not been authenticated.

When the interconnecting device 40 receives through its interconnecting port 42 from the management device 10 a message that the IC card 30 has been authenticated, the control part 41 allows the patient using this IC card 30 to use the network 100 as described later. On the other hand, when the interconnecting device 40 receives through the communication port 47 from the management device 10 a message that the IC card 30 has been authenticated, the control part 41 prohibits the patient with the IC card 30 from using the network 100, and indicates a message “the IC card has not been authenticated” on the display part 48.

A duty to use of the IC card 30 may exclude a person who does not have it from using the network 100, and prevents an authorized use of the network 100. For example, a person without the IC card 30 cannot apply for a medical treatment. In addition, even a patient who was once registered in the personal information database 15a in the storage part 15 cannot use the network 100 in some cases, for example, where he has an outstanding bill, and where his health insurance expires. The patient whose IC card 30 has not been authenticated may inquire the administrator through the reception 210e to request him to reissue a valid IC card 30 so as to solve this problem.

On the other hand, when the IC card 30 has been authenticated, the interconnecting device 40d indicates a menu screen for providing the patient with desired information, and for obtaining a request from the user (step 1006). For example, the control part 41 indicates four options including “1. waiting time”, “2. application for today's medical treatment”, “3. reservation of medical treatment”, “4. end” in the menu screen on the display part 48. As discussed, the control part 41 may additionally indicate other information on to an availability and status of use of facilities in the hospital 200 and service associated with the facility, for example, including an availability of each doctor in a specific date and time, a relationship between symptoms and proper departments, a description of a prescription medicine, and other frequent asked questions.

The “waiting time” option provides the current waiting time for a medical treatment for each department. The “application for today's medical treatment” option used for a registration of a today's medical treatment. This option works as if the patient submits the consultation ticket to the reception. The “reservation of medical treatment” option is used for a reservation of a medical treatment. The “end” option finishes the menu screen. This menu screen is an initial screen in this embodiment. Of course, items to be included in the menu screen are not limited to the above items. For example, the menu screen may include various options, such as “change of reservation”, “cancel of reservation”, “doctor” to designate a specific doctor or female doctors for female patients, and “symptoms” to assist in a selection of a proper department by entering a patient's symptoms.

The patient selects a desired item in the menu items, and specifies it using ten key 49a in the input part 49 in the interconnecting device 40d. In response, the control part 41 recognizes a signal (i.e., a pulse signal corresponding to a numeral) sent from the input part 49, and moves to the item selected by the patient.

A description will now be given of operations of the interconnecting device 40d for each menu item.

When the patient presses the number 1 and selects an indication of the waiting time, the interconnecting device 40d then recites plural departments in the menu screen so as to prompt the patient to select one department among them. For example, the control part 41 indicates seven options including “1. internal department”, “2. department of surgery”, “3. department of inspection”, “4. pharmacy”, “5. reception or cashier”, “6. return”, and “7. end” on the display part 48. The first five options are used for a selection of a department by the patient. The “return” option switches the current screen to the previous screen on the display part 48. The “end” option finishes the procedure, and returns to the initial screen. Similarly, the patient selects a desired item from the menu items by inputting it into the interconnecting device 40 using ten key 49a in the input part 49.

If the patient wishes to display the waiting time for the internal department 210a and presses the number 1, the interconnecting device 40d receives the waiting information (i.e., the number of waiting persons and the estimated waiting time) from the interconnecting device 40a provided at the internal department 210a.

A description will now be given of an exemplary calculation of the waiting time by the interconnecting device 40 in the management system 1. Initially, the administrator arbitrarily determines the average time arbitrarily and stores an average medical treatment time per one patient in the ROM 44 or the storage part 45. For example, the average medical treatment time is fifteen minutes. The control part 41 calculates the waiting time taking the number of registering and reserving patients into consideration (step 1008).

For example, when it is 9:00 a.m. now and there are ten registering patients and two patients who have reservations at 10:15 a.m. and 14:30 a.m., the timetable 45a assigns five registering patients from 9:00 a.m. to 10:15 a.m., and other five registering patients from 10:30 a.m. to 11:45 a.m. taking the patient who has a reservation at 10:15 a.m. into consideration. As a result, the control part 45 may determine three hours the waiting time to 12:00 a.m. based on the timetable 45a and the current time. Whenever each patient finishes his medical treatment, the control part 45 updates the waiting time by actual medical treatment time. In other words, the control part 45 may correct the waiting time using an error between fifteen minutes and the actual medical treatment time, although a detailed description of the correction method will be omitted here.

When the patient presses the number 1 and select the waiting time of the internal department, the control part 41 in the interconnecting device 40 sends, through its interconnecting port 42, a request for waiting information to the interconnecting device 40a in the internal department 210a. The interconnecting device 40a that has received the request through its interconnecting port 42 from the interconnecting device 40d sends, through its interconnecting port 42, the waiting information, which is calculated by the above manner, to the interconnecting device 40d. As a result, the control part 41 in the interconnecting device 40d receives the waiting information through the interconnecting port 42.

When the interconnecting device 40d receives the waiting information, it indicates the estimated waiting time and/or the number of waiting patients for the internal department on the display part 48 (step 1010). For example, the control part 41 in the interconnecting part 40d may indicate the waiting information including “internal department”, “the number of waiting persons, “estimated waiting time”, as shown in FIG. 7, and three options including “1. application for today's medical treatment”, “2. reservation of medical treatment”, and “3. end” on the display part 48. These three options are the same as those discussed for the initial menu screen, and a description thereof will be omitted. Thereby, the patient recognizes the waiting time for a medical treatment, and then manipulates the input part 49 to apply for today's medical treatment (by pressing number “1”) or make a reservation for a medical treatment on a different day (by pressing number “2”).

When the patient selects “6. return” by pressing the number 6 through ten key 49a in the input part 49, the control part 41 switches to the initial menu screen on the display part 48. When the patient selects “7. end” by pressing number 7 through ten key 49a in the input part 49, the control part 41 switches to the initial menu screen on the display part 48.

When the patient selects “2. application for today's medical treatment” after the step 1006 by pressing the number 2, the interconnecting device 40d prompts the patient to select a desired department in the next screen. For example, the control part 41 indicates seven options including “1. internal department”, “2. department of surgery”, “3. department of inspection”, “4. pharmacy”, “5. reception or cashier”, “6. return”, and “7. end” on the display part 48. These options are the same as those described above, and a detailed description thereof will be omitted. The patient selects a desired item from the menu items and inputs it through ten key 49a in the input part 49. This menu screen may be omitted when the patient has already selected a specific department (for example, the internal department 210a), displayed the waiting time, and applied for today's medical treatment (i.e., by entering the number 1 using the ten key 49a after the waiting information was displayed on the screen).

When the patient selects the internal department 210a by entering the number 1 and wishes to apply for today's medical treatment in the internal department 210a, the control part 41 requests the interconnecting device 40a in the internal department 210a to register the patient's application for the today's medical treatment. When receiving, through the interconnecting port 42, the request from the interconnecting device 40d, the interconnecting device 40a assigns (the name and ID of) the patient to the timetable 45a in the possible earliest turn in its storage part 45 (step 1012) and then stores the information in the timetable 45a in the storage part 45 (step 1014). The control part 41 in the interconnecting device 40a notifies the interconnecting device 40d through the interconnecting port 42 of the acceptance of the request with the assigned time of the medical treatment. The control part 41 in the interconnecting device 40d receives such information through the interconnecting port 42.

When the control part 41 in the interconnecting device 40d obtains the acceptance information, the control part 41 indicates the fixed registration information for the patient on the display part 48 (step 1016). For example, the control part 41 in the interconnecting part 40d may indicate the registration information including “internal department”, “request for application accepted”, “reservation date and time: Jun. 10, 2001, 14:00 p.m.”, and an option “1. end” on the display part 48. The option “end” is the same as that discussed for the initial menu screen, and a description thereof will be omitted. Optionally, the control part 41 further indicates an option that enables the patient to enter his symptoms including a headache, fever, allergy to a medicine for the doctor's review.

When the patient uses the interconnecting device 40 in front of the target department (for example, the interconnecting device 40a in the internal department 210a) to apply for a today's medical treatment, the control part 41 in the interconnecting part 40a assigns (the name and ID of) the patient to the timetable 45a in the possible earliest turn in its storage part 45 (step 1012) and then stores the information in the timetable 45a in the storage part 45 (step 1014). In order to fix the registration, the control part 41 in the interconnecting device 40a indicates the fixed registration information for the patient on the display part 48 (step 1016). For example, the control part 41 in the interconnecting part 40d may indicate the registration information including “internal department”, “request for application accepted”, “reservation date and time: Jun. 10, 2001, 14:00 p.m.”, and an option “1. end” on the display part 48. The option “end” is the same as that discussed for the initial menu screen, and a description thereof will be omitted. Optionally, the control part 41 further indicates an option that enables the patient to enter his symptoms including a headache, fever, allergy to a medicine for the doctor's review. Thereby, the patient finishes applying for today's medical treatment.

When the patient selects “6. return” by pressing the number 6 through ten key 49a in the input part 49, the control part 41 switches to the initial menu screen on the display part 48. When the patient selects “7. end” by pressing number 7 through ten key 49a in the input part 49, the control part 41 switches to the initial menu screen on the display part 48.

When the patient selects “3. reservation of medical treatment” after the step 1006 by pressing the number 3, the interconnecting device 40d prompts the patient to select a desired department in the next screen. For example, the control part 41 indicates seven options including “1. internal department”, “2. department of surgery”, “3. department of inspection”, “4. pharmacy”, “5. reception or cashier”, “6. return”, and “7. end” on the display part 48. These options are the same as those described above, and a detailed description thereof will be omitted. The patient selects a desired item from the menu items and inputs it through ten key 49a in the input part 49. This menu screen may be omitted when the patient has already selected a specific department (for example, the internal department 210a), displayed the waiting time, and reserved a medical treatment (i.e., by entering the number 2 using the ten key 49a after the waiting information was displayed on the screen).

When the patient wishes to reserve a medical treatment in the internal department 210a by entering the number 1, then he enters the specific date, e.g., Dec. 5, 2001. For example, the control part 41 indicates a message “enter the date by six digits” on the display part 48, and prompts the patient to enter “120501” representative of Dec. 5, 2001 using the ten key 49a in the input part 49. Alternatively, the patient may specify a range of period of time, e.g., Dec. 5, 2001 and Dec. 6, 2001. Then, the control part 41 sends the patient's request for reservation with the specific date to the interconnecting device 40a.

When receiving, through the interconnecting port 42, the request from the interconnecting device 40d, the interconnecting device 40a sends back to the interconnecting device 40d a list of available time on the specific date or a message that the specific date is unavailable after searching using the timetable 45a. For example, the interconnecting device 40a sends back to the interconnecting device 40d the reservation information including “internal department”, “specified date: Dec. 5, 2001”, “available time: 14:00 to 16:00”, and two options “1. select” and “2. end”. After reviewing the reservation information, the patient wishes to specify, for example, the desired time from 14:00 to 16:00 by pressing the number 1. Then, the control part 41 indicates a message “enter the date by six digits” on the display part 48, and prompts the patient to enter “1430” representative of 14:30 p.m., for example, using the ten key 49a in the input part 49. If the patient does not like the time range from 14:00 to 16:00, he would select “end” option to change the date.

After the patient specifies the time, the control part 41 sends to the interconnecting device 40a a request for reservation of a medical treatment at 14:30 p.m. When receiving, through the interconnecting port 42, the request from the interconnecting device 40d, the interconnecting device 40a assigns (the name and ID of) the patient to the specified date and time in the timetable 45a in its storage part 45 (step 1012) and then stores the information in the timetable 45a in the storage part 45 (step 1014). The control part 41 in the interconnecting device 40a notifies the interconnecting device 40d through the interconnecting port 42 of the acceptance of the request. The control part 41 in the interconnecting device 40d receives such information through the interconnecting port 42.

When the control part 41 in the interconnecting device 40d obtains the acceptance information, the control part 41 indicates the fixed reservation information for the patient on the display part 48 (step 1016). For example, the control part 41 in the interconnecting part 40d may indicate the reservation information including “internal department”, “request for reservation accepted”, “reservation date and time: Jun. 10, 2001, 14:00 p.m.”, and an option “1. end” on the display part 48. The option “end” is the same as that discussed for the initial menu screen, and a description thereof will be omitted. Optionally, the control part 41 further indicates an option that enables the patient to enter his symptoms including a headache, fever, allergy to a medicine for the doctor's review.

When the patient uses the interconnecting device 40 in front of the target department (for example, the interconnecting device 40a in the internal department 210a) to reserve a medical treatment, then he enters the specific date, as discussed above. Then, the control part 41 in interconnecting device 40a displays a list of available time on the specific date or a message that the specific date is unavailable after searching in the timetable 45a, as discussed above. After reviewing the reservation information, the patient specifies a desired time or terminates the screen to change the date. When the patient specifies the time, the interconnecting device 40a assigns (the name and ID of) the patient to the specified date and time in the timetable 45a in its storage part 45 (step 1012) and then stores the information in the timetable 45a in the storage part 45 (step 1014). Then, the control part 41 may indicate the registration information including “internal department”, “request for application accepted”, “reservation date and time: Dec. 5, 2001, 14:30 p.m.”, and an option “1. end” on the display part 48. Optionally, the control part 41 further indicates an option that enables the patient to enter his symptoms including a headache, fever, allergy to a medicine for the doctor's review. The option “end” is the same as that discussed for the initial menu screen, and a description thereof will be omitted.

When the patient selects “4. end” after the step 1006 by pressing the number 4 through the ten key 49a in the input part 49, the control part 41 switches the menu screen to the initial screen on the display part 48.

As understood from the above description of the interconnecting device 40, the patient may know the waiting information of each department, for example, by using the interconnecting device 40d at the reception 210e. Since he may make a registration of a today's medical treatment, he may maximize the time to the medical treatment, for example, coming back home, finishing his business, and visiting another department's medical treatment after confirming the waiting time at the other department. In addition, the users may obtain and utilize such information by themselves, and thus the management system 1 is laborsaving for the hospital 200.

Before going to another department, the patient may grasp the number of waiting persons and the estimated waiting time using the interconnecting device 40. Thereby, the patient may secure his turn for a medical treatment at the next department and go there after confirming the waiting information.

Before the medical treatment, the patient submits his IC card 30 to a doctor. The doctor checks the chart history referring to the IC card 30 using the network device 70 and the IC card drive 80. After the medical treatment, the doctor writes chart information (including information on diagnosis and prescription) into the patient's IC card 30 using the network device 70 and the IC card drive 80, and returns it to the patient for his review. At the same time, the chart information may be automatically stored in the personal information database 15a so as to share the patient's chart information through the network devices 70 in the hospital 200. The chart information is also used for the doctor at the next medical treatment. The IC card 30 serves as a chart, and eliminates various troubles including an erroneous diagnosis caused by a nurse who has taken a different patient's chart. In addition, a nurse does not have to deliver a patient's chart from the department to a cashier, saving the time necessary for the delivery and reducing the patient's waiting time.

Before paying the bill at the cashier, the patient may grasp the number of waiting persons and the estimated waiting time using the interconnecting device 40. Thereby, the patient may register his turn for the payment at the cashier and go there after confirming the waiting information. In some case, the patient uses an automatic deduction from his bank account or an automatic charge to his credit card account for payment, and avoids lining up at the cashier.

Before going to the pharmacy or dispensary to receive a prescription medicine, the patient may grasp the number of waiting persons and the estimated waiting time using the interconnecting device 40. Thereby, the patient may secure his turn at the pharmacy and go there after confirming the waiting information. A pharmacist uses the network device 70 and IC card drive 80 provided in the pharmacy 210d to supply the prescription medicine in accordance with the prescription information stored in the IC card 30. Alternatively, the prescription information may be automatically sent from the interconnecting device 40a to the interconnecting device 40c when the doctor in the internal department 210a stores the chart information into the IC card 30. In this case, the pharmacist may prepare for the prescription in advance to the patient's visit.

Thus, the instant embodiment provides a patient with information to utilize the facilities in the hospital 200 and services associated with the facilities, and enables the patient to reserve a medical treatment and to apply for a today's medical treatment using the interconnecting device 40. The management device 10 provides a centralized management to the network 100 and patients' information. The management system 1 is laborsaving and timesaving for the management of the hospital 200. A patient may grasp the availability and a status of use of each facility in the hospital 200, and spend his time to the best advantage.

As stated above, a patient may use the terminal 90 in addition to the interconnecting device 40. When the terminal 90 is a portable terminal, such as a cellular phone, or a PC provided in a user's house, the terminal 90 may be connected to the network 100 through an Internet, etc. and the communication port 47 in the interconnecting device 40. Using the terminal 90, the patient access the network 100 from a remote location, such as his home, his company, and the outside. Such a system also enables the patient to spend his time to best advantage by saving patient's time to go to the hospital 200. In addition, the instant management system is used to provide a patient with a description (e.g., ingredient) of a prescription medicine that has nothing to do with the waiting information.

Further, the present invention is not limited to the preferred embodiment, and various variations and modifications may be made without departing from the present invention. For example, the present invention is applicable to a bank, a restaurant, a municipal office, a health club, a beauty salon, and the like.

According to the inventive management system, a user of a facility, such as a hospital, may grasp the availability and status of use of the facility and service associated with the facility, and provides the user with efficient utilization of the facility. For example, a patient of a hospital confirms the waiting time information of a target department in advance, and then registers or reserves a medical examination or treatment and efficiently uses the waiting time.

What is claimed is:

1. A management system for managing a facility that is available to users, said management system comprising:
a network provided in the facility and connectible to a terminal;
a communication part for communicating with the terminal through the network;
a first memory for storing first information on an availability and status of use of the facility and service associated with the facility, the first information being able to be updated; and
a control part for allowing the user of the terminal to access first information.

2. A management system according to claim 1, further comprising:
a second memory for storing second information on identifications of the users; and
an authentication part for authenticating a person who uses the terminal, based on the second information in the second memory and third information entered by the person through the terminal, wherein said control part allows the person to access the first information when said authentication part authenticates the person.

3. A management system according to claim 2, wherein each user has an information record carrier for storing the third information, and the terminal includes a drive for reading the third information from the information record carrier.

4. A management system according to claim 3, wherein the information record carrier stores fourth information on a result of use of the facility and service associated with the facility.

5. A management system according to claim 4, wherein the users are patients and the fourth information includes a chart.

6. A management system according to claim 1, wherein the third information includes a condition of the user.

7. A management system according to claim 6, wherein the users are patients, and the condition of the user includes symptoms.

8. A management system according to claim 1, wherein said control part accepts a reservation, a cancellation of the reservation and a change of the reservation by the user of the facility and service associated with the facility, and updates the first information to reflect the reservation, the cancellation of the reservation, and the change of the reservation.

9. A management system according to claim 1, wherein each user has an information record carrier for storing fourth information on a result of use of the facility and service associated with the facility by the user, and
wherein the management system further comprises:
a drive for writing the fourth information in the information record carrier after use of the facility and service associated with the facility by the user;
a cashier part for creating billing information based on the fourth information written by said drive.

10. A management system according to claim 9, further comprising a third memory for storing a table that correlates expenses and use of facility and service associated with the facility, wherein said cashier part automatically calculates the billing information referring to said third memory.

11. A management system according to claim 9, wherein the information record carrier further stores fifth information on an account of the user, and said cashier part bills the account based on the fifth information.

12. A management system according to claim 1, wherein each user is a patient and has an information record carrier, the information record carrier storing fourth information on a result of a medical treatment and a prescription medicine, the facility including a pharmacy,
wherein the management system further comprises a drive, provided in the pharmacy, for reading the fourth information in the information record carrier to provide the prescription medicine.

13. A management system according to claim 1, wherein the first information is selected from among the number of waiting persons and/or estimated waiting time for each department in a hospital in the specific date and time, an availability of each doctor in a specific date and time, an availability of each facility in the hospital in the specific date and time, a relationship between symptoms and proper departments, a description of a prescription medicine, and frequent asked questions.

14. A management system according to claim 1, wherein said control part controls connection status and traffic of said network.

15. A management system according to claim 1, wherein the terminal logs in the network in a wireless manner.

16. A management method for managing a facility that is available to users, said management method comprising the steps of:
storing first information on an availability and status of use of the facility and service associated with the facility by the users, the first information being able to be updated momentarily and accessible to each of the users through a terminal and an information record carrier that stores the third information;
storing second information on identification of the users;
authenticating a person who uses the terminal, based on the second information and third information entered by the person through the terminal and the information record carrier; and
allowing the person to access the first information when said authentication step authenticates the person.

17. A management method according to claim 16, further comprising the steps of:
accepting a reservation, a cancellation of the reservation and a change of the reservation by the person to reserve the facility and service associated with the facility on a specific date and time when said authentication step authenticates the person; and
updating the first information to reflect the reservation, the cancellation of the reservation, and the change of the reservation.

18. A management device connected to a network provided in a facility that is available to users, and a terminal through the network, said network being connected to a first memory for storing first information on an availability and status of use of the facility and service associated with the facility, the first information being able to be updated momentarily, said management device comprising:
a second memory for storing second information on identification of the users;
an authentication part for authenticating a person who uses the terminal, based on the second information in the second memory and third information entered by the person through the terminal, and
a control part allows the person to access the first information in the first memory when said authentication part authenticates the person.

19. A program that enables a computer to execute a management method for managing a facility that is available to users, said management method comprising the steps of:
storing first information on an availability and status of use of the facility and service associated with the facility by the users, the first information being able to be updated momentarily and accessible to each of the users through a terminal and an information record carrier that stores the third information; and
storing second information on identification of the users;
authenticating a person who uses the terminal, based on the second information and third information entered by the person through the terminal and the information record carrier; and
allowing the person to access the first information when said authentication step authenticates the person.
